(19) United States
(12) Patent Application Publication
(10) Pub. No.: US 2017/0312439 A1
(43) Pub. Date: Nov. 2, 2017

(54) Syringe Devices and Methods

(71) Applicant: Kimberly Y. Kelley, Arab, AL (US)

(72) Inventor: Kimberly Y. Kelley, Arab, AL (US)

(21) Appl. No.: 15/142,101

(22) Filed: Apr. 29, 2016

(51) Int. Cl. A61M 5/315; A61M 5/31

(52) U.S. Cl. CPC A61M 5/31511; A61M 2005/3126; A61M 2005/31516

(57) ABSTRACT

A syringe of the present disclosure has a hollow barrel fixedly and removeably coupled at one end to a needle and having an orifice at a second end. The hollow barrel also has a calibration inscription inscribed on an outer surface for identifying volumetric quantities of fluid being delivered within the hollow barrel, and the calibration inscription is a first color. The syringe of the present disclosure further has a plunger that has a compressible bulb coupled to an end of the plunger, and the compressible plunger has a second color that is different than the first color of the calibration inscription.

### BACKGROUND

Medical professionals, for example nurses, doctors, and dentists, use medical devices that inject fluid into, or withdraw fluid from, the body. These types of devices are commonly referred to as syringes.

A typical syringe generally comprises a hollow barrel on which there are volumetric indicators inscribed. As an example, a syringe may have indicators in measurements of millimeters or cubic centimeters (CCs). The volumetric indicators are often referred to “calibrations.”

Additionally, a hollow needle may be fixedly coupled to the hollow barrel through which fluids may be injected or withdrawn. Other types of delivery devices may be affixed to the hollow barrel in other types of applications. For example, the hollow barrel may be fixedly coupled to a tube, often temporarily inserted into the body for the delivery of a substantial and continuous flow of fluids.

Further, the syringe comprises a plunger that is insertable and slidably coupled to the hollow barrel. The plunger comprises a plunger shaft that is coupled at one end to a flat end tip or thumb rest for grasping the plunger. Additionally, a rubber bulb is fixedly coupled to an opposing end. The rubber bulb comprises an air-tight seal that ensures that fluid being drawn into the hollow barrel does not escape the orifice of the hollow barrel.

In use, the plunger is inserted into an orifice providing slidable entry into the hollow barrel. For delivery of fluids to the body, the plunger is pushed to the opposing end of the hollow barrel, and fluid is drawn into the hollow barrel as the plunger is pulled, but not removed, from the hollow barrel. This action draws fluid into the hollow barrel. In order to determine the amount of fluid being drawn into the hollow barrel, the medical professional pulls the plunger and visually inspects the calibration inscriptions. When the rubber bulb moveably coincides with the calibration inscription indicating the desired volume, the medical professional ceases pulling the plunger from the hollow barrel. In the example provided wherein the syringe is to be used to deliver fluids to the body, the medical professional inserts the needle into a patient's body and pushes the plunger in a direction toward the needles, and fluid exits the needle and enters, for example, a vein, in the patient's body.

Note that a traditional syringe comprises calibrations that are inscribed on the hollow body in black. Also, the traditional syringe comprises a rubber bulb that is also black. It is often difficult when in use to discern the volumetric indication desired because the black rubber bulb and the black calibration inscriptions visually mesh.

### SUMMARY

A syringe of the present disclosure has a hollow barrel fixedly and removeably coupled at one end to a needle and having an orifice at a second end, the hollow barrel has a calibration inscription inscribed on an outer surface for identifying volumetric quantities of fluid being delivered within the hollow barrel, and the calibration inscription being a first color. Additionally, the syringe has a plunger having a compressible bulb coupled to an end of the plunger, wherein the compressible plunger has a second color that is different than the first color of the calibration inscription.

### BRIEF DESCRIPTION OF THE DRAWINGS

The present disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1A is a side view of an exemplary syringe in accordance with an embodiment of the present disclosure showing a plunger of the syringe that has been pushed into a barrel of the syringe.

FIG. 1B is an end view of the syringe of FIG. 1A taken along line A-N.

FIG. 2 is a side view of the exemplary syringe depicted in FIG.1A showing a plunger of the syringe that has been pulled and removed from the barrel.

### DETAILED DESCRIPTION

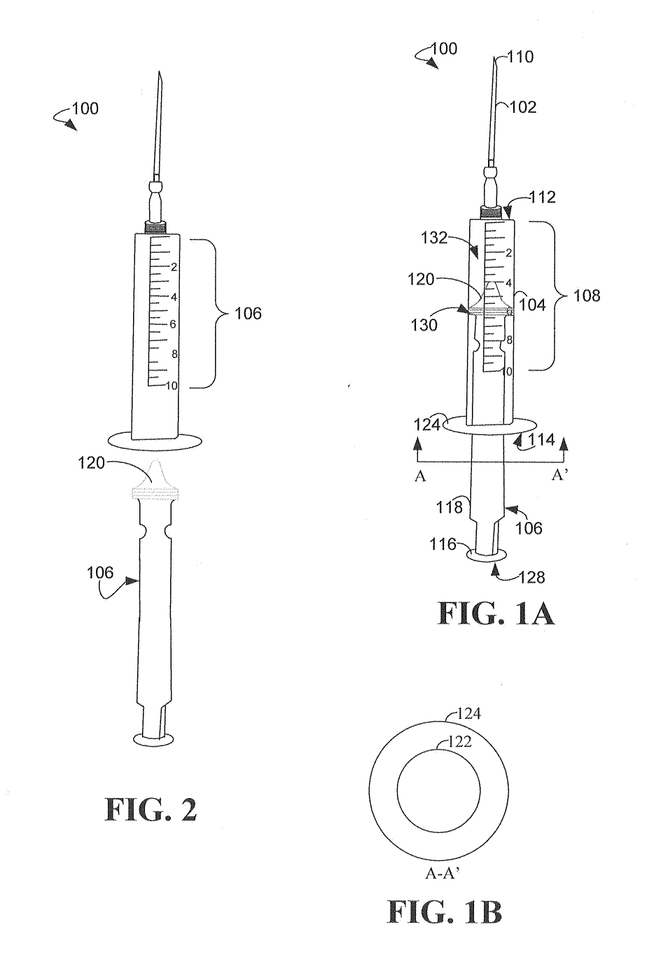

The present disclosure describes an exemplary syringe that provides easier readability for a medical professional using the exemplary syringe. In this regard, the exemplary syringe comprises a needle coupled to a hollow tube. In an exemplary embodiment, the hollow tube is inscribed with calibrations for indicating volumetric amounts for identifying fluid volume to be drawn in and thereafter delivered to a patient's vein. Note that this use is merely exemplary. There are other uses in other embodiments for the syringe of the present disclosure. The syringe further comprises a plunger having a rubber bulb coupled to one end and a thumb rest on the other. The syringe of the present disclosure comprises calibrations inscribed on the hollow barrel; however, the inscription is a color that is different that than from the color of the rubber bulb. As a mere example, the calibration inscriptions may be black and the rubber bulb may be a pastel color. Thus, as fluid is being drawn into the hollow barrel via the plunger, the medical professional more easily sees the demarcations of the calibration inscriptions. Thus, the medical professional more easily knows when the correct amount of fluid has been drawn into the hollow barrel. FIG. 1A depicts a syringe 100 in accordance with an embodiment of the present disclosure. The syringe comprises a hollow barrel 104. A needle 102 is fixedly and removeably coupled to an end 112 of the hollow barrel, and on an opposing end 114 is a finger flange 124.

Note that the needle 102 may be affixed to the hollow barrel via one or more devices, for example a luer slip, a luer lock, or an irrigation tip. Any other type of device known in the art or future-developed may be used to affix the needle to the hollow barrel 104.

With reference to FIG. 1B, in one embodiment, the finger flange 124 extends outwardly for use in actuation of the syringe 100. Further, the hollow barrel 104 comprises an orifice 122 through which a plunger 106 is inserted. Note that other types of components may be affixed to the opposing end 114 of the syringe 100 in other embodiments of the present disclosure.

The hollow barrel 104 is transparent. The transparency allows a medical professional who is using the syringe 100 to view the plunger 106 as fluid is being drawn into the hollow barrel.

Note that the present disclosure does not comprise potentially limiting description of the dimensions of the hollow barrel 104. The present disclosure anticipates any dimensions of the hollow barrel would come under the purview of the present disclosure.

In this regard, any dimensions known in the art or future-developed of the hollow barrel 104 may be used in manufacture of the syringe 100. As mere examples, the inside diameter of the hollow barrel 104 may be 6/32″, 11/32″, 16/32″, 9/16′, ¾″, ⅞″, or ⅛″. Other sizes may be used for the hollow barrel 104 in other embodiments.

The plunger 106 comprises a plunger shaft 118. On one end 128 of the plunger shaft 118 is a thumb rest 116. Note that other devices for aiding actuation of the plunger may be used in other embodiments of the present disclosure.

Further, a compressible bulb 120 is fixedly coupled to an opposing end 130 of the plunger shaft 118. Note that the bulb may be made of rubber, for example; however, the bulb 120 may be made of other force compressible materials in other embodiments of the present disclosure. The compressible bulb 120 acted upon by the inner walls of the hollow barrel 104 as the plunger is being inserted into the hollow barrel 104 applies an opposing force to the inner walls ensuring an airtight and watertight coupled to the inner walls so that fluid does not escape from the hollow barrel 104 as fluid is drawn in, contained, and delivered to a patient's body via a needle 102 comprising a orifice 110 for delivery.

Note that a particular shape of compressible bulb 120 is shown in FIG. 1A; however other types of shapes or forms of the compressible bulb 120 may be used in other embodiments of the present disclosure. In this regard, any type of component that provides compression to ensure that fluid in the hollow barrel does not escape is possible in other embodiments.

The syringe 100 further comprises calibration inscription 108 on an outer transparent surface 132 of the hollow barrel 104. In this regard, the calibration inscription 108 comprises volumetric identifiers, e.g., 2, 4, 6, 8, 10. The identifiers indicate a volume of fluid (or gas) that is being delivered within the hollow barrel 104 when the plunger is actuated. In this regard, when the compressible bulb 120 aligns with one of the identifiers, the alignment indicates the volume of fluid that is in the hollow barrel 104. The medical professional using the syringe 100 may be able to view the vertical movement of the compressible bulb 120 through the transparent hollow barrel 104.

In one embodiment, the calibration inscription 108 is a particular color, e.g., black. In such an embodiment, in accordance with an aspect of the present disclosure, the compressible bulb 120 is a color different than color of the calibration inscription 108. In the example wherein the particular color is black, the compressible bulb 120 may be, for example, a lighter color. As an example, the compressible bulb 120 may be a pastel color. Pastel colors may include pastel pink, green, blue, yellow, or purple. In another embodiment, the color of the compressible bulb may be a primary color, e.g., red, blue, or yellow. In yet another embodiment, the compressible bulb 120 may be any variant of the pastel color or the primary color, so long as the color of the compressible bulb 120 is different than the color of the calibration inscription 108. The differing color of the compressible bulb 108 ensures that the medical professional using the syringe 100 can more easily read the calibration inscription 108 in order to determine that the hollow barrel 104 is sufficiently filled. In this regard, the differentiation between the color of the calibration inscription 108 and the color of the compressible bulb 120 ensures that the medical professional using the syringe 100 may easily discern the volumetric reading because the color of the calibration inscription 108 does not visually interfere with the color of the compressible bulb 120.

Note that pastel colors and primary colors are specifically mentioned above. The present disclosure relates to any color of compressible plunger 120 that is different that the color of the calibration inscription 108. The present disclosure is not intended to be limiting in this regard. As an example, the calibration inscription may be a color other than black, e.g., blue. In such a scenario, the color of the compressible bulb 120 is any color other than blue that the medical professional can differentiate between the calibration inscription 108 and the compressible bulb 120. In the scenario in which the calibration inscription 108 is blue, the compressible bulb 120 may be, for example, pink.

FIG. 2 is a side view of the syringe 100 showing the plunger 106 removed from the hollow barrel 104. This particular view in FIG. 2 is shown to further describe the plunger 106. In this regard, the calibration inscription 108 on the surface 132 of the hollow barrel 104 is a particular color. For example, the calibration inscription 108 may be black. In such an embodiment, the compressible bulb 120 is a color different that the particular color of the calibration inscription 108.

In the example provided wherein the calibration inscription is black, the compressible bulb 120 is a color other than black. For example, the compressible bulb 120 may be a pastel color, as described herein. In addition, the compressible bulb 120 may be one of the primary colors, as described herein. Thus, when the medical professional using the syringe tries to determine the volumetric indicator of the calibration inscription 108, the color of the calibration inscription 108 does not interfere with the color of the compressible bulb 120.

What is claimed is:

1. A syringe, comprising:
a hollow barrel fixedly and removeably coupled at one end to a needle and having an orifice at a second end, the hollow barrel comprising a calibration inscription inscribed on an outer surface for identifying volumetric quantities of fluid being delivered within the hollow barrel, the calibration inscription being a first color; and
a plunger having a compressible bulb coupled to an end of the plunger, wherein the compressible plunger has a second color that is different than the first color of the calibration inscription.

2. The syringe of claim 1, wherein the first color is black.

3. The syringe of claim 2, wherein the second color is a color different than black.

4. The syringe of claim 2, wherein the second color is a pastel color.

5. The syringe of claim 4, wherein the pastel color is one selected from the group consisting of pastel pink, pastel green, pastel blue, pastel yellow, or pastel orange.

6. The syringe of claim 2, wherein the second color is a primary color.

7. The syringe of claim 6, wherein the primary color is one selected from the group consisting of primary red, primary blue, or primary green.
